(19) United States
(12) Patent Application Publication
(10) Pub. No.: US 2004/0101879 A1
(43) Pub. Date: May 27, 2004

(54) Srebp pathway modulation through targeting hisrs

(76) Inventors: Cynthia Seidel-Dugan, Benicia, CA (US); Lisa C. Kadyk, San Francisco, CA (US); Felipa A. Mapa, San Francisco, CA (US)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 10/466,161

(22) PCT Filed: Jan. 11, 2002
(86) PCT No.: PCT/US02/01045

(51) Int. Cl. C12Q 1/68; G01N 33/53; G01N 33/567

(52) U.S. Cl. 435/6; 435/7.2

(57) ABSTRACT

Human HisRS genes are identified as modulators of the SREPB pathway and thus are therapeutic for disorders associated with the SREBP pathway. Methods for identifying modulators of HisRS, comprising screening for agents that modulate the activity of HisRS are provided.

This application claims priority to U.S. Provisional Application No. 60/261,569, filed on Jan. 12, 2001, which is incorporated by reference in its entirety.

### BACKGROUND OF THE INVENTION

There is much interest within the pharmaceutical industry to understand the mechanisms involved in cholesterol synthesis and metabolism, particularly on the molecular level, so that blood cholesterol lowering drugs can be developed for the treatment or prevention of atherosclerosis. There is further interest in understanding the molecular mechanisms that connect lipid defects and insulin resistance. Hyperlipidemia and elevation of free fatty acid levels correlate with “Metabolic Syndrome,” defined as the linkage between several diseases, including obesity and insulin resistance, which often occur in the same patients and which are major risk factors for development of Type 2 diabetes and cardiovascular disease. Current research suggests that the control of lipid levels, in addition to glucose levels, may be required to treat Type 2 Diabetes, heart disease, and other manifestations of Metabolic Syndrome (Santomauro A T et al., Diabetes (1999) 48:1836-1841).

Recent advances have been made in understanding some of the mechanisms involved in mammalian lipid metabolism. A key component is the sterol regulatory element binding protein (SREBP) pathway. SREBPs are transcription factors that activate genes involved in cholesterol and fatty acid synthesis and transport. SREBP is the major mediator of insulin action in the liver, and alterations in expression and function of SREBPs have been described in obese and insulin resistant patients or animal models (Shimomura I et al., PNAS (1999) 96:13656-61; Shimomura I et al., Journal of Biological Chemistry (1999) 274:30028-32). SREBPs are also implicated in the process of fat cell differentiation and adipose cell gene expression, particularly as transcription factors that can promote adipogenesis in a dominant fashion (reviewed by Spiegelman et al., Cell (1996) 87:377-389). SREBP function is regulated by intracellular levels of sterols or polyunsaturated fatty acids (PUFAs) (Xu J. et al., J. Biol. Chem. (1999) 274:23577-23583).

In high sterol or PUFA conditions, SREBPs are retained as membrane-bound protein precursors that are kept inactive by virtue of being attached to the nuclear envelope and endoplasmic reticulum (ER) and therefore, excluded from the nucleus. An SREBP in its membrane-bound form has large N-terminal and C-terminal segments facing the cytoplasm and a short loop projecting into the lumen of the organelle. The N-terminal domain is a transcription factor of the basic-helix-loop-helix-leucine zipper (bHIH-Zip) family, and contains an “acid blob” typical of many transcriptional activators (Brown and Goldstein, Cell (1997) 89:331-340). The N-terminal acid blob is followed by a basic helix-loop-helix/leucine zipper domain (bHLH-Zip) similar to those found in many other DNA-binding transcriptional regulators.

Several components of the SREBP signaling pathway are known. In low sterol conditions, the acid blob/bHLH-Zip domain of SREBP is released from the membrane after which it is rapidly translocated into the nucleus and binds specific DNA sequences to activate transcription. Two sequential proteolytic cleavages are involved. A first protease, referred to as the site 1 protease (S1P) cleaves SREBP at approximately the middle of the lumenal loop (Sakai et al., J. Biol. Chem (1998) 273:5785-5793).

After cleavage at site 1, a second protease, the site 2 protease (S2P) cleaves the N-terminal fragment and releases the mature N-terminal domain into the cytosol, from which it rapidly enters the nucleus, apparently with a portion of the transmembrane domain still attached at the C-terminus (Rawson et al., Molec Cell (1997) 1:47-57). Mature, transcriptionally active SREBP is rapidly degraded in a proteosome-dependent process. This combination of proteolytic processing and rapid turnover allows the SREBP system to rapidly respond to changes in cellular membrane components.

A third component of the processing system for SREBPs is called SREBP Cleavage Activating Protein (SCAP). SCAP is a large transmembrane protein that activates S I P in low-sterol conditions (Hua et al., Cell (1996) 87:415-426). To date, the SREBP pathway has been studied primarily using mammalian cell culture, by the isolation of mutant cells that are defective in regulation of cholesterol metabolism or intracellular cholesterol trafficking. The mutants can then serve as hosts for cloning genes by functional complementation. This has led to the molecular cloning of the S1P, S2P and SCAP genes (Rawson et al., supra; Hua et al., supra; Goldstein et al., U.S. Pat. Nos. 5,527,690 and 5,891,631 and PCT Application No. WO00/09677).

Relatively little is known about additional processing proteins of the SREBP pathway and about regulation of their activation. Proteins that regulate SREBP function might be excellent therapeutic targets for controlling dyslipidemia and the associated increased risk for cardiovascular disease.

Some SREBP pathway genes have been identified in invertebrates. The isolation of a Drosophila SREBP, referred to as “HLH106” (GI079656) has been described (Theopold et al., Proc. Natl. Acad. Sci., U.S.A, (1996) 93(3): 1195-1199). The identification of the C. elegans SREBP, as well as other Drosophila and C. elegans SREBP pathway genes is disclosed in WO00076308A1.

The ability to manipulate and screen the genomes of model organisms such as Drosophila and C. elegans provides a powerful means to analyze biochemical processes that, due to significant evolutionary conservation of genes, pathways, and cellular processes, have direct relevance to more complex vertebrate organisms. Identification of novel functions of genes involved in particular pathways in such model organisms can directly contribute to the understanding of the correlative pathways in mammals and of methods of modulating them (see e.g., Miklos G L and Rubin G M, Cell 1996, 86:521-529). While Drosophila and C. elegans are not susceptible to human pathologies, various experimental models can mimic the pathological states. A correlation between the pathology model and the modified expression of a Drosophila or C. elegans gene can identify the association of the human orthologue with the human disease.

In one example, a genetic screen is performed in an invertebrate model organism displaying a mutant (generally visible or selectable) phenotype due to misexpression—generally reduced, enhanced or ectopic expression—of a known gene (the “genetic entry point”). Additional genes are mutated in a random or targeted manner. When an additional gene mutation changes the original mutant phenotype, this gene is identified as a “modifier” that directly or indirectly interacts with the genetic entry point and its associated pathway. If the genetic entry point is an ortholog of a human gene associated with a human pathology, such as lipid metabolic disorders, the screen can identify modifier genes that are candidate targets for novel therapeutics.

In a screen using C. elegans SREBP as a genetic entry point, we discovered that T11G6.1 (GenBank Identifier [GI] 7507690), the C. elegans ortholog of the human histidyl tRNA synthetase (HisRS) gene (GI 6996014), is modifier of SREBP function.

The primary function of histidyl tRNA synthetases (HisRS) is to attach histidine to histidine tRNA (tRNA^{His}), which is essential for incorporation of histidine into proteins. HisRS proteins are homodimeric enzymes that have characteristic motifs shared by class II tRNA synthetases (also including Gly, Pro, Ser and Thr tRNA synthetases). These enzymes are conserved throughout evolution, in species ranging from bacteria to humans (Amaar, Y. G. and D. L. Baillie (1993) Nucl. Acids Res. 221: 4344-4347). In addition to their primary function, HisRS is one of several aaRS that can synthesize the intracellular signalling molecule diadenosine tetraphosphate (Freist, W. et al. (1999) Biol. Chem. 380: 623-646; Kisselev, et al. (1998) FEBS Lett. 427: 157-163). This molecule is abundant in pancreatic G-cells, and is believed to inhibit ATP-sensitive K+ (KATP) channels (Jovanovic, et al. (1997) Biochemical Pharmacology 54: 219-225). In pancreatic cells, inhibition of these channels is necessary for glucose-stimulated insulin release (this is the mechanism of action of sulfonylureas).

In addition, HisRS has been implicated in nutrient sensing and control of translation through regulation of p70 S6Kinase. When activated, S6K phosphorylates the 40S ribosomal protein S6 which results in the increased initiation of translation of ribosomal mRNAs (Kawasome, et al. (1998) Proc. Natl. Acad. Sci. U.S.A 95: 5033-5038). It was shown that cells bearing a temperature-sensitive mutation in HisRS, when shifted to non-permissive temperature, have reduced S6K activity (Iiboshi, Y., et al. (1999) J. Biol Chem. 274: 1092-1099). The mechanism of this feedback is not understood, but it appears that accumulation of deacylated tRNAs results in a signal which inhibits S6K activity.

### SUMMARY OF THE INVENTION

The invention provides a method of identifying candidate agents for modulating the SREBP pathway, lipid metabolism, and/or adipogenesis using an assay system comprising a HisRS polypeptide or nucleic acid; contacting the assay system with a test agent under conditions whereby, but for the presence of the test agent, the system provides a reference activity; and detecting a test agent-biased activity of the assay system, wherein a difference between the test agent-biased activity and the reference activity identifies the test agent as a candidate agent for modulating the SREBP pathway, lipid metabolism, and/or adipogenesis. Candidate test agents include small molecule modulators, antibodies, and nucleic acid modulators such as antisense oligomers and PMOs, among others.

In one embodiment of the invention, the assay system comprises cultured cells or a non-human animal expressing HisRS, and the assay system detects an agent-biased change in the SREBP pathway, lipid metabolism, and/or adipogenesis.

In certain embodiments, candidate HisRS-modulating agents are identified in cell-free or cell-based assays, and a second assay system that detects an agent-biased change in an activity associated with the SREBP pathway, lipid metabolism, and/or adipogenesis is used to confirm the SREBP pathway modulating activity of the candidate agent. In a preferred embodiment, the second assay detects an agent-biased change in an activity associated with SREBP pathway. Preferred second assay systems are carried out in cultured cells.

The invention further provides a method of modulating the SREBP pathway in a mammalian cell comprising contacting the cell with an agent that specifically binds a HisRS polypeptide or nucleic acid. In a preferred embodiment, the agent is administered to a mammalian animal predetermined to have a pathology associated with the SREBP pathway. Preferred agents include small molecule modulators, nucleic acid modulators, or antibodies.

### DETAILED DESCRIPTION OF THE INVENTION

We used a C. elegans model for defective SREBP signaling to identify the association of HisRS with the SREBP pathway. We have shown that reduction or elimination of SREBP activity in C. elegans (through gene mutation or RNA interference [RNAi]) results in a pale intestine phenotype due to reduced lipid accumulation in the intestine. In addition, strong loss-of-function of C. elegans SREBP causes a larval arrest phenotype (see WO00076308A1). A genetic screen was performed in C. elegans to identify modifiers (suppressors) of the SREBP pale intestine phenotype. Specifically, we used an RNAi feeding system to produce the loss-of-function SREBP phenotype, and screened for genes that when mutated result in restoration of a more normal phenotype. C. elegans that were fed E. coli producing double stranded (ds) RNA corresponding to the SREBP gene (“on SREBP RNAi feeding plates”) displayed the pale intestine phenotype. For the screen, wild-type worms were mutagenized with EMS, allowed to self-fertilize for two generations to generate worms homozygous for newly induced mutations. The resulting homozygotes were placed on SREBP RNAi feeding plates. C. elegans containing suppressor mutations were identified as having intestines that were more darkly pigmented that the pale intestines of wild type C. elegans on SREBP RNAi feeding plates. The modifier gene's identity was discovered through genetic mapping, positional cloning and phenotypic rescue of mutants.

We identified T11G6.1(GI 7507690), an ortholog of the human HisRS gene, as a suppressor of the C. elegans SREBP phenotype, and hence a member of the SREBP pathway. Accordingly, HisRS genes (i.e., nucleic acids and polypeptides) are attractive drug targets for the treatment of disorders related to lipid (e.g., fatty acid and cholesterol) metabolism, adipogenesis, and/or other pathologies associated with the SREBP signaling pathway. In one example, treatment involves decreasing signaling through the SREBP pathway in order to treat pathologies related to metabolic syndrome.

The invention provides in vitro and in vivo methods of assessing HisRS function, and methods of modulating (generally inhibiting or agonizing) HisRS activity, which are useful for further elucidating the SREBP pathway and for developing diagnostic and therapeutic modalities for pathologies associated with the SREBP pathway. As used herein, pathologies associated with the SREBP signaling pathway encompass pathologies where the SREBP pathway contributes to maintaining the healthy state, as well as pathologies whose course may be altered by modulation of the SREBP pathway.

### HisRS Nucleic Acids and Polypeptides

Human HisRS nucleic acid (cDNA) and protein sequences are provided in SEQ ID NOs: 1 and 2, respectively.

The term “HisRS polypeptide” refers to a full-length HisRS protein or a fragment or derivative thereof that is “functionally active,” meaning that the HisRS protein derivative or fragment exhibits one or more functional activities associated with a full-length, wild-type HisRS protein. As one example, a fragment or derivative may have antigenicity such that it can be used in immunoassays, for immunization, for generation of inhibitory antibodies, etc, as discussed further below. Preferably, a functionally active HisRS fragment or derivative displays one or more biological activities associated with HisRS proteins such as enzymatic activity, signaling activity, ability to bind natural cellular substrates, etc. Preferred HisRS polypeptides display enzymatic activity. In one embodiment, a functionally active HisRS polypeptide is a HisRS derivative capable of rescuing defective endogenous HisRS activity, such as in cell based or animal assays; the rescuing derivative may be from the same or a different species. If HisRS fragments are used in assays to identify modulating agents, the fragments preferably comprise a HisRS domain, such as a C- or N-terminal or catalytic domain, among others, and preferably comprise at least 10, preferably at least 20, more preferably at least 25, and most preferably at least 50 contiguous amino acids of a HisRS protein. Functional domains can be identified using the PFAM program (Bateman A et al., 1999 Nucleic Acids Res 27:260-262; website at pfam.wustl.edu).

The term “HisRS nucleic acid” refers to a DNA or RNA molecule that encodes a HisRS polypeptide. Preferably, the HisRS polypeptide or nucleic acid or fragment thereof is from a human, but it can be an ortholog or derivative thereof with at least 70%, preferably with at least 80%, preferably 85%, still more preferably 90%, and most preferably at least 95% sequence identity with human HisRS. As used herein, “percent (%) sequence identity” with respect to a specified subject sequence, or a specified portion thereof, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al., J. Mol. Biol. (1997) 215:403-410; http://blast.wustl.edu/blast/README.html) with search parameters set to default values. The HSP S and HSP S2 parameters are dynamic values and are established by the program itself depending upon the composition of the particular sequence and composition of the particular database against which the sequence of interest is being searched. A “% identity value” is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation. A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Derivative nucleic acid molecules of the subject nucleic acid molecules include sequences that hybridize to the nucleic acid sequence of SEQ ID NO:1. The stringency of hybridization can be controlled by temperature, ionic strength, pH, and the presence of denaturing agents such as formamide during hybridization and washing. Conditions routinely used are set out in readily available procedure texts (e.g., Current Protocol in Molecular Biology, Vol. 1, Chap. 2.10, John Wiley & Sons, Publishers (1994); Sambrook et al., Molecular Cloning, Cold Spring Harbor (1989)). In some embodiments, a nucleic acid molecule of the invention is capable of hybridizing to a nucleic acid molecule containing the nucleotide sequence of SEQ ID NO:1 under stringent hybridization conditions that comprise: prehybridization of filters containing nucleic acid for 8 hours to overnight at 65° C. in a solution comprising 6× single strength citrate (SSC) (1×SSC is 0.15 M NaCl, 0.015 M Na citrate; pH 7.0), 5× Denhardt's solution, 0.05% sodium pyrophosphate and 100 μg/ml herring sperm DNA; hybridization for 18-20 hours at 65° C. in a solution containing 6×SSC, 1× Denhardt's solution, 100 μg/ml yeast tRNA and 0.05% sodium pyrophosphate; and washing of filters at 65° C. for 1 h in a solution containing 0.2×SSC and 0.1% SDS (sodium dodecyl sulfate). In other embodiments, moderately stringent hybridization conditions are used that comprise: pretreatment of filters containing nucleic acid for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH 7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS. Alternatively, low stringency conditions can be used that comprise: incubation for 8 hours to overnight at 37° C. in a solution comprising 20% formamide, 5×SSC, 50 mM sodium phosphate (pH 7.6), 5× Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

In some embodiments, the HisRS is an ortholog of human HisRS. Methods of identifying the human orthologs of these genes are known in the art. Normally, orthologs in different species retain the same function, due to presence of one or more protein motifs and/or 3-dimensional structures. Orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen M A and Bork P, Proc Natl Acad Sci (1998) 95:5849-5856; Huynen M A et al., Genome Research (2000) 10:1204-1210). Programs for multiple sequence alignment, such as CLUSTAL (Thompson J D et al, 1994, Nucleic Acids Res 22:4673-4680) may be used to highlight conserved regions and/or residues of orthologous proteins and to generate phylogenetic trees. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. In evolution, when a gene duplication event follows speciation, a single gene in one species, such as Drosophila, may correspond to multiple genes (paralogs) in another, such as human. As used herein, the term “orthologs” encompasses paralogs.

### Isolation, Production, Expression, and Mis-expression of HisRS Nucleic Acids and Polypeptides

HisRS nucleic acids and polypeptides are useful for identifying and testing agents that modulate HisRS function and for other applications related to the involvement of HisRS in the SREBP pathway. HisRS nucleic acids may be obtained using any available method. For instance, techniques for isolating cDNA or genomic DNA sequences of interest by screening DNA libraries or by using polymerase chain reaction (PCR) are well known in the art.

A wide variety of methods are available for obtaining HisRS polypeptides. In general, the intended use for the polypeptide will dictate the particulars of expression, production, and purification methods. For instance, production of polypeptides for use in screening for modulating agents may require methods that preserve specific biological activities of these proteins, whereas production of polypeptides for antibody generation may require structural integrity of particular epitopes. Expression of polypeptides to be purified for screening or antibody production may require the addition of specific tags (i.e., generation of fusion proteins). Overexpression of a HisRS polypeptide for cell-based assays used to assess HisRS function, such as involvement in lipid metabolism, may require expression in eukaryotic cell lines capable of these cellular activities. Techniques for the expression, production, and purification of proteins are well known in the art; any suitable means therefor may be used (e.g., Higgins S J and Hames B D (eds.) Protein Expression: A Practical Approach, Oxford University Press Inc., New York 1999; Stanbury P F et al., Principles of Fermentation Technology, 2^{nd} edition, Elsevier Science, New York, 1995; Doonan S (ed.) Protein Purification Protocols, Humana Press, New Jersey, 1996; Coligan J E et al, Current Protocols in Protein Science (eds.), 1999, John Wiley & Sons, New York; U.S. Pat. No. 6,165,992).

The nucleotide sequence encoding a HisRS polypeptide can be inserted into any appropriate vector for expression of the inserted protein-coding sequence. The necessary transcriptional and translational signals, including promoter/enhancer element, can derive from the native HisRS gene and/or its flanking regions or can be heterologous. A variety of host-vector expression systems may be utilized, such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid, or cosmid DNA. A host cell strain that modulates the expression of, modifies, and/or specifically processes the gene product may be used.

The HisRS polypeptide may be optionally expressed as a fusion or chimeric product, joined via a peptide bond to a heterologous protein sequence. In one application the heterologous sequence encodes a transcriptional reporter gene (e.g., GFP or other fluorescent proteins, luciferase, beta-galactosidase, etc.). A chimeric product can be made by ligating the appropriate nucleic acid sequences encoding the desired amino acid sequences to each other in the proper coding frame using standard methods and expressing the chimeric product. A chimeric product may also be made by protein synthetic techniques, e.g. by use of a peptide synthesizer (Hunkapiller et al., Nature (1984) 310:105-111).

A HisRS polypeptide can be isolated and purified using standard methods (e.g. ion exchange, affinity, and gel exclusion chromatography; centrifugation; differential solubility; electrophoresis). Alternatively, native HisRS proteins can be purified from natural sources, by standard methods (e.g. immunoaffinity purification). Once a protein is obtained, it may be quantified and its activity measured by appropriate methods, such as immunoassay, bioassay, or other measurements of physical properties, such as crystallography.

The methods of this invention may also use cells that have been engineered for altered expression (mis-expression) of HisRS or other genes associated with the SREBP pathway, lipid metabolism, and/or adipogenesis. As used herein, mis-expression encompasses ectopic expression, over-expression, under-expression, and non-expression (e.g. by gene knock-out or blocking expression that would otherwise normally occur).

### Genetically Modified Animals

The methods of this invention may use non-human animals that have been genetically modified to alter expression of HisRS and/or other genes known to be involved in adipogenesis, lipid metabolism, and/or the SREBP pathway. Preferred genetically modified animals are mammals, particularly mice or rats. Preferred non-mammalian species include Zebrafish, C. elegans, and Drosophila. Preferably, the altered HisRS or other gene expression results in a detectable phenotype, such as modified lipid profile as compared to control animals having normal expression of the altered gene. The genetically modified animals can be used to further elucidate the SREBP pathway, in animal models of pathologies associate with adipogenesis, lipid metabolism, and/or the SREBP pathway, and for in vivo testing of candidate therapeutic agents, as described below.

Preferred genetically modified animals are transgenic, at least a portion of their cells harboring non-native nucleic acid that is present either as a stable genomic insertion or as an extra-chromosomal element, which is typically mosaic. Preferred transgenic animals have germ-line insertions that are stably transmitted to all cells of progeny animals.

Non-native nucleic acid is introduced into host animals by any expedient method. Methods of making transgenic non-human animals are well-known in the art (for mice see Brinster et al., Proc. Nat. Acad. Sci. U.S.A 1985, 82:4438-42; U.S. Pat. Nos. 4,736,866, 4,870,009, 4,873,191, 6,127,598; Hogan, B., Manipulating the Mouse Embryo, Cold Spring Harbor Laboratory Press, Cold Spring Harbor, N.Y., (1986); for homologous recombination see Capecchi, Science 1989, 244:1288-1292; Joyner et al., Nature1989, 338:153-156; for particle bombardment see U.S. Pat. No., 4,945,050; for Drosophila see Rubin and Spradling, Science (1982) 218:348-53, U.S. Pat. No. 4,670,388; for transgenic insects see Berghammer A. J. et al., Nature 1999, 402:370-371; for Zebrafish see Lin S. Methods Mol Biol. (2000);136:375-3830; for fish, amphibians and birds see Houdebine and Chourrout, Experientia (1991) 47:897-905; for rats see Hammer et al., Cell (1990)63:1099-1112; for embryonic stem (ES) cells see Teratocarcinomas and Embryonic Stem Cells, A Practical Approach, E. J. Robertson, ed., IRL Press (1987); for livestock see Pursel et al., Science (1989) 244:1281-1288; for nonhuman animal clones see Wilmut, I. et al. (1997) Nature 385:810-813, PCT Publication Nos. WO 97/07668 and WO 97/07669; for recombinase systems for regulated transgene expression see, Lakso et al., PNAS (1992) 89:6232-6236; U.S. Pat. No. 4,959,317 [for cre.loxP] and O'Gorman et al. (1991) Science 251:1351-1355; U.S. Pat. No. 5,654,182 [for FLP/FRT]).

Homozygous or heterozygous alterations in the genomes of transgenic animals may result in mis-expression of native genes, including ectopic expression, over-expression (e.g. by multiple gene copies), under-expression, and non-expression (e.g. by gene knock-out or blocking expression that would otherwise normally occur). In one application, a “knock-out” animal is generated, typically using homologous recombination, in which an alteration in an endogenous gene causes a decrease in that gene's function, preferably such that gene expression is undetectable or insignificant.

### HisRS Modulating Agents

The invention provides methods to identify agents that interact with and/or modulate the function of HisRS and/or the SREBP pathway. Such agents are useful in a variety of diagnostic and therapeutic applications associated with the SREBP pathway, as well as in further analysis of the HisRS protein and its contribution to the SREBP pathway. Accordingly, the invention also provides methods for modulating the SREBP pathway comprising the step of specifically modulating HisRS activity by administering a HisRS-interacting or -modulating agent.

In a preferred embodiment, HisRS-modulating agents inhibit or enhance HisRS activity or otherwise affect normal HisRS function, including transcription, protein expression, protein localization, and cellular or extra-cellular activity. In a further preferred embodiment, the candidate SREBP pathway-modulating agent specifically modulates the function of the HisRS. The phrases “specific modulating agent”, “specifically modulates”, etc., are used herein to refer to modulating agents that directly bind to the HisRS polypeptide or nucleic acid, and preferably inhibit, enhance, or otherwise alter the function of the HisRS. The term also encompasses modulating agents that alter the interaction of HisRS with a binding partner or substrate (e.g. by binding to a binding partner of a HisRS, or to a protein/binding partner complex, and inhibiting function).

Preferred HisRS-modulating agents include small molecule chemical agents, HisRS-interacting proteins, including antibodies and other biotherapeutics, and nucleic acid modulators, including antisense oligomers and RNA. The modulating agents may be formulated in pharmaceutical compositions, for example, as compositions that may comprise other active ingredients, as in combination therapy, and/or suitable carriers or excipients. Techniques for formulation and administration of the compounds may be found in “Remington's Pharmaceutical Sciences” Mack Publishing Co., Easton, Pa., 19^{th} edition.

### Small Molecule Modulators

Chemical agents, referred to in the art as “small molecule” compounds are typically organic, non-peptide molecules, having a molecular weight less than 10,000, preferably less than 5,000, more preferably less than 1,000, and most preferably less than 500. This class of modulators includes chemically synthesized molecules, for instance, compounds from combinatorial chemical libraries. Synthetic compounds may be rationally designed or identified based on known or inferred properties of the HisRS protein or may be identified by screening compound libraries. Alternative appropriate modulators of this class are natural products, particularly secondary metabolites from organisms such as plants or fungi, which can also be identified by screening compound libraries for HisRS-modulating activity. Methods for generating and obtaining compounds are well known in the art (Schreiber S L, Science (2000) 151: 1964-1969; Radmann J and Gunther J, Science (2000) 151:1947-1948).

Small molecule modulators identified from screening assays, as described below, can be used as lead compounds from which candidate clinical compounds may be designed, optimized, and synthesized. Such clinical compounds may have utility in treating pathologies associated with the SREBP pathway. The activity of candidate small molecule modulating agents may be improved several-fold through iterative secondary functional validation, as further described below, structure determination, and candidate modulator modification and testing. Additionally, candidate clinical compounds are generated with specific regard to clinical and pharmacological properties. For example, the reagents may be derivatized and re-screened using in vitro and in vivo assays to optimize activity and minimize toxicity for pharmaceutical development.

### Protein Modulators

A HisRS-interacting protein may be endogenous, i.e. one that normally interacts genetically or biochemically with a HisRS, such as a member of the HisRS pathway that modulates HisRS expression, localization, and/or activity. HisRS-modulators include dominant negative forms of HisRS-interacting proteins and of HisRS proteins themselves. Yeast two-hybrid and variant screens offer preferred methods for identifying endogenous HisRS-interacting (Finley, R. L. et al. (1996) in DNA Cloning-Expression Systems: A Practical Approach, eds. Glover D. & Hames B. D (Oxford University Press, Oxford, England), pp. 169-203; Fashema S F et al., Gene (2000) 250:1-14; Drees B L Curr Opin Chem Biol (1999) 3:64-70; Vidal M and Legrain P Nucleic Acids Res (1999) 27:919-29; and U.S. Pat. No. 5,928,868). Mass spectrometry offers alternative preferred methods for the elucidation of protein complexes (reviewed in, e.g., Pandley A and Mann M, Nature (2000) 405:837-846; Yates J R ₃rd, Trends Genet (2000) 16:5-8). A HisRS -interacting protein may also be a T-cell antigen receptor (Harlow and Lane, 1988, supra).

### Antibody Modulators

In a preferred embodiment, the HisRS-interacting protein is an antibody. Antibodies that specifically bind HisRS polypeptides can be generated using known methods. Preferably the antibody is specific to a mammalian HisRS polypeptide, and more preferably, a human HisRS. Antibodies may be polyclonal, monoclonal (mAbs), humanized or chimeric antibodies, single chain antibodies, Fab fragments, F(ab′).sub.2 fragments, fragments produced by a FAb expression library, anti-idiotypic (anti-Id) antibodies, and epitope-binding fragments of any of the above. Monoclonal antibodies with affinities of 10⁸ M⁻¹ preferably 10⁹ M⁻to 10¹⁰ M⁻¹, or stronger can be made by standard procedures as described (Harlow E and Lane D, 1988, Antibodies: A Laboratory Manual, CSH Laboratory Press; (Harlow E and Lane D, 1999 Using Antibodies: A Laboratory Manual, CSH Laboratory Press; Goding (1986) Monoclonal Antibodies: Principles and Practice (2d ed) Academic Press, New York; and U.S. Pat. Nos. 4,381,292; 4,451,570; and 4,618,577). Antibodies may be generated against crude cell extracts of HisRS or substantially purified fragments thereof. If HisRS fragments are used, they preferably comprise at least 10, and more preferably, at least 20 contiguous amino acids of a HisRS protein. In a particular embodiment, HisRS-specific antigens and/or immunogens are coupled to carrier proteins that stimulate the immune response. For example, the subject polypeptides are covalently coupled to the keyhole limpet hemocyanin (KLH) carrier, and the conjugate is emulsified in Freund's complete adjuvant, which enhances the immune response. An appropriate immune system such as a laboratory rabbit or mouse is immunized according to conventional protocols.

Chimeric antibodies specific to HisRS polypeptides can be made that contain different portions from different animal species. For instance, a human immunoglobulin constant region may be linked to a variable region of a murine mAb, such that the antibody derives its biological activity from the human antibody, and its binding specificity from the murine fragment. Chimeric antibodies are produced by splicing together genes that encode the appropriate regions from each species (Morrison et al., Proc. Natl. Acad. Sci. (1984) 81:6851-6855; Neuberger et al., Nature (1984) 312:604-608; Takeda et al., Nature (1985) 31:452-454). Humanized antibodies, which are a form of chimeric antibodies, can be generated by grafting complementary-determining regions (CDRs) (Carlos, T. M., J. M. Harlan. 1994. Blood 84:2068-2101) of mouse antibodies into a background of human framework regions and constant regions by recombinant DNA technology (Riechmann L M, et al., 1988 Nature 323: 323-327). Humanized antibodies contain ˜10% murine sequences and ˜90% human sequences, and thus further reduce or eliminate immunogenicity, while retaining the antibody specificities (Co MS, and Queen C. 1991 Nature 351:501-501; Morrison S L. 1992 Ann. Rev. Immun. 10:239-265). Humanized antibodies and methods of their production are well-known in the art (U.S. Pat. No. 5,530,101; U.S. Pat. No. 5,585,089; U.S. Pat. No. 5,693,762, and U.S. Pat. No. 6,180,370).

HisRS-specific single chain antibodies, which are recombinant, single chain polypeptides formed by linking the heavy and light chain fragments of the Fv regions via an amino acid bridge, can be produced (U.S. Pat. No. 4,946,778; Bird, Science (1988) 242:423-426; Huston et al., Proc. Natl. Acad. Sci. U.S.A (1988) 85:5879-5883; and Ward et al., Nature (1989) 334:544-546).

Other suitable techniques for antibody production involve in vitro exposure of lymphocytes to the antigenic polypeptides or alternatively to selection of libraries of antibodies in phage or similar vectors (Huse et al., Science (1989) 246:1275-1281).

As used herein, T-cell antigen receptors are included within the scope of antibody modulators (Harlow and Lane, 1988, supra).

The polypeptides and antibodies of the present invention may be used with or without modification. Frequently, the polypeptides and antibodies will be labeled by joining, either covalently or non-covalently, a substance that provides for a detectable signal, or that is toxic to cells that express the targeted protein (Menard S, et al., Int J. Biol Markers (1989) 4:131-134). A wide variety of labels and conjugation techniques are known and are reported extensively in both the scientific and patent literature. Suitable labels include radionuclides, enzymes, substrates, cofactors, inhibitors, fluorescent moieties, fluorescent emitting lanthanide metals, chemiluminescent moieties, bioluminescent moieties, magnetic particles, and the like (U.S. Pat. Nos. 3,817,837; 3,850,752; 3,939,350; 3,996,345; 4,277,437; 4,275,149; and 4,366,241). Also, recombinant immunoglobulins may be produced (U.S. Pat. No. 4,816,567). Antibodies to cytoplasmic proteins may be delivered and reach their targets by conjugation with membrane-penetrating toxin proteins (U.S. Pat. NO. 6,086,900).

When used therapeutically in a patient, the antibodies of the subject invention are typically administered parenterally, when possible at the target site, or intravenously. The therapeutically effective dose and dosage regimen is determined by clinical studies. Typically, the amount of antibody administered is in the range of about 0.1 mg/kg-to about 10 mg/kg of patient weight. For parenteral administration, the antibodies are formulated in a unit dosage injectable form (e.g., solution, suspension, emulsion) in association with a pharmaceutically acceptable vehicle. Such vehicles are inherently nontoxic and non-therapeutic. Examples are water, saline, Ringer's solution, dextrose solution, and 5% human serum albumin. Nonaqueous vehicles such as fixed oils, ethyl oleate, or liposome carriers may also be used. The vehicle may contain minor amounts of additives, such as buffers and preservatives, which enhance isotonicity and chemical stability or otherwise enhance therapeutic potential. The antibodies' concentrations in such vehicles are typically in the range of about 1 mg/ml-to about 10 mg/ml. Immunotherapeutic methods are further described in the literature (U.S. Pat. No. 5,859,206; WO0073469).

### Nucleic Acid Modulators

Other preferred HisRS-modulating agents comprise nucleic acid molecules, such as antisense oligomers or double stranded RNA (dsRNA), which generally inhibit HisRS activity.

Preferred antisense oligomers interfere with the function of HisRS nucleic acids, such as DNA replication, transcription, HisRS RNA translocation, translation of protein from the HisRS RNA, RNA splicing, and any catalytic activity in which the HisRS RNA participates. In one embodiment, the antisense oligomer is an oligonucleotide that is sufficiently complementary to a HisRS mRNA to bind to and prevent translation from the HisRS mRNA, preferably by binding to the 5′ untranslated region. HisRS-specific antisense oligonucleotides preferably range from at least 6 to about 200 nucleotides. In some embodiments the oligonucleotide is preferably at least 10, 15, or 20 nucleotides in length. In other embodiments, the oligonucleotide is preferably less than 50, 40, or 30 nucleotides in length. The oligonucleotide can be DNA or RNA, a chimeric mixture of DNA and RNA, derivatives or modified versions thereof, single-stranded or double-stranded. The oligonucleotide can be modified at the base moiety, sugar moiety, or phosphate backbone. The oligonucleotide may include other appending groups such as peptides, agents that facilitate transport across the cell membrane, hybridization-triggered cleavage agents, and intercalating agents.

In another embodiment, the antisense oligomer is a phosphorothioate morpholino oligomer (PMO). PMOs are assembled from four different morpholino subunits, each of which containing one of four genetic bases (A, C, G, or T) linked to a six-membered morpholine ring. Polymers of these subunits are joined by non-ionic phosphodiamidate inter-subunit linkages. Methods of producing and using PMOs and other antisense oligonucleotides are well known in the art (e.g. see W099/18193; Summerton J, and Weller D, Antisense Nucleic Acid Drug Dev 1997, 7:187-95; Probst J C, Methods 2000, 22:271-281; U.S. Pat. No. 5,325,033; U.S. Pat. No. 5,378,841).

Antisense oligomers are commonly used as research reagents, diagnostics, and therapeutics. For example, antisense oligonucleotides, which are able to specifically inhibit gene expression, are often used to elucidate the function of particular genes (see, e.g., U.S. Pat. No. 6,165,790). Antisense oligomers are also used, for example, to distinguish between functions of various members of a biological pathway. Antisense oligomers have been employed as therapeutic moieties in the treatment of disease states in animals and humans and have been demonstrated in numerous clinical trials to be safe and effective (Milligan J F et al, 1993, J Med Chem 36:1923-1937; Tonkinson J L et al., 1996, Cancer Invest 14:54-65). Accordingly, in one aspect of the invention, a HisRS-specific antisense oligomer is used in an assay to further elucidate the function of HisRS in the SREBP pathway. In another aspect of the invention, a HisRS-specific antisense oligomer is used as a therapeutic agent for treatment of metabolic pathologies.

Alternative preferred HisRS-modulating agents are double-stranded RNA species mediating RNA interference (RNAi). RNAi is the process of sequence-specific, post-transcriptional gene silencing in animals and plants, initiated by double-stranded RNA (dsRNA) that is homologous in sequence to the silenced gene. Methods relating to the use of RNAi to silence genes in C. elegans, Drosophila, plants, and mammals are known in the art (Fire A, et al., 1998 Nature 391:806-811; Fire, A. Trends Genet. 15, 358-363 (1999); Sharp, P. A. RNA interference 2001. Genes Dev. 15,485-490 (2001); Hammond, S. M., et al., Nature Rev Genet 2, 110-1119 (2001); Tuschl, T. Chem Biochem. 2, 239-245 (2001); Hamilton, A. et al., Science 286, 950-952 (1999); Hammond, S. M., et al., Nature 404, 293-296 (2000); Zamore, P. D., et al., Cell 101, 25-33 (2000); Bernstein, E., et al., Nature 409, 363-366 (2001); Elbashir, S. M., et al., Genes Dev. 15, 188-200 (2001); WO0129058; WO9932619, and Elbashir S M, et al., 2001, Nature 411:494-498).

### Assay Systems

The invention provides assay systems for identifying specific modulators of HisRS activity. As used herein, an “assay system” encompasses all the components required for performing and analyzing results of an assay that detects and/or measures a particular event or events. In general, primary assays are used to identify or confirm a modulator's specific biochemical or molecular effect with respect to the HisRS nucleic acid or protein. In general, secondary assays further assess the activity of a HisRS-modulating agent identified by a primary assay and may confirm that the modulating agent affects HisRS in a manner relevant to the SREBP pathway, lipid metabolism and/or adipogenesis. In some cases, HisRS-modulators will be directly tested in a “secondary assay,” without having been identified or confirmed in a “primary assay.”

In a preferred embodiment, the assay system comprises contacting a suitable assay system comprising a HisRS polypeptide or nucleic acid with a candidate agent under conditions whereby, but for the presence of the agent, the system provides a reference activity, which is based on the particular molecular event the assay system detects. The method further comprises detecting the same type of activity in the presence of a candidate agent (” the agent-biased activity of the system”). A difference between the agent-biased activity and the reference activity indicates that the candidate agent modulates HisRS activity, and hence the SREBP pathway. A difference, as used herein, is statistically significant. The assay systems generally include positive and/or negative controls, as are well known in the art.

### Primary Assays

The type of modulator tested generally determines the type of primary assay.

### Primary Assays for Small Molecule Modulators

For small molecule modulators, screening assays are used to identify candidate modulators. Screening assays may be cell-based or may use a cell-free system that recreates or retains the relevant biochemical reaction of the target protein (reviewed in Sittampalam G S et al., Curr Opin Chem Biol (1997) 1:384-91 and accompanying references). As used herein the term “cell-based” refers to assays using live cells, dead cells, or a particular cellular fraction, such as a membrane, endoplasmic reticulum, or mitochondrial fraction. The term “cell free” encompasses assays using substantially purified protein (either endogenous or recombinantly produced), partially purified cellular extracts, or crude cellular extracts. Screening assays may detect a variety of molecular events, including protein-DNA interactions, protein-protein interactions (e.g., receptor-ligand binding), transcriptional activity (e.g., using a reporter gene), enzymatic activity (e.g., via a property of the substrate), activity of second messengers, immunogenicty and changes in cellular morphology or other cellular characteristics. Appropriate screening assays may use a wide range of detection methods including fluorescent, radioactive, colorimetric, spectrophotometric, and amperometric methods, to provide a read-out for the particular molecular event detected.

In a preferred embodiment, screening assays use fluorescence technologies, including fluorescence polarization, time-resolved fluorescence, and fluorescence resonance energy transfer. These systems offer means to monitor protein-protein or DNA-protein interactions in which the intensity of the signal emitted from dye-labeled molecules depends upon their interactions with partner molecules (e.g., Selvin P R, Nat Struct Biol (2000) 7:730-4; Fernandes P B, Curr Opin Chem Biol (1998) 2:597-603; Hertzberg R P and Pope A J, Curr Opin Chem Biol (2000) 4:445-451).

Suitable assay formats that may be adapted to screen for HisRS modulators are known in the art. A primary function of HisRS is to attach histidine to histidine-tRNA. Accordingly, suitable assays may detect aminoacylation activity of this enzyme (e.g., Yan W et al., 1996, Biochemistry 35:6559-68). Preferred screening assays are high throughput or ultra high throughput and thus provide automated, cost-effective means of screening compound libraries for lead compounds (Fernandes P B, 1998, supra; Sundberg S A, Curr Opin Biotechnol 2000, 11:47-53).

Cell-based screening assays usually require systems for recombinant expression of HisRS and any auxiliary proteins demanded by the particular assay. Cell-free assays often use recombinantly produced purified or substantially purified proteins. Appropriate methods for generating recombinant proteins produce sufficient quantities of proteins that retain their relevant biological activities and are of sufficient purity to optimize activity and assure assay reproducibility. Yeast two-hybrid and variant screens, and mass spectrometry provide preferred methods for determining protein-protein interactions and elucidation of protein complexes. In certain applications when HisRS-interacting proteins are used in screening assays, the binding specificity of the interacting protein to the HisRS protein may be assayed by various known methods, including binding equilibrium constants (usually at least about 10⁷ M⁻¹, preferably at least about 10⁸ M⁻¹, more preferably at least about 10⁹ M⁻), and immunogenic properties. For enzymes and receptors, binding may be assayed by, respectively, substrate and ligand processing.

The screening assay may measure a candidate agent's ability to specifically bind to or modulate activity of a HisRS polypeptide, a fusion protein thereof, or to cells or membranes bearing the polypeptide or fusion protein. The HisRS polypeptide can be full length or a fragment thereof that retains functional HisRS activity. The HisRS polypeptide may be fused to another polypeptide, such as a peptide tag for detection or anchoring, or to another tag. The HisRS polypeptide is preferably human HisRS, or is an ortholog or derivative thereof as described above. In a preferred embodiment, the screening assay detects candidate agent-based modulation of HisRS interaction with a binding target, such as an endogenous or exogenous protein or other substrate that has HisRS-specific binding activity, and can be used to assess normal HisRS gene function.

Certain screening assays may also be used to test antibody and nucleic acid modulators; for nucleic acid modulators, appropriate assay systems involve HisRS mRNA expression.

### Primary Assays for Antibody Modulators

For antibody modulators, appropriate primary assays are binding assays that test the antibody's affinity to and specificity for the HisRS protein. Methods for testing antibody affinity and specificity are well known in the art (Harlow and Lane, 1988, 1999, supra). The enzyme-linked immunosorbant assay (ELISA) is a preferred methods for detecting HisRS-specific antibodies, others include FACS assays, radioimmunoassays, and fluorescent assays.

### Primary Assays for Nucleic Acid Modulators

For nucleic acid modulators, primary assays may test the ability of the nucleic acid modulator to inhibit HisRS gene expression, preferably mRNA expression. In general, expression analysis comprises comparing HisRS expression in like populations of cells (e.g., two pools of cells that endogenously or recombinantly express HisRS) in the presence and absence of the nucleic acid modulator. Methods for analyzing mRNA and protein expression are well known in the art. For instance, Northern blotting, slot blotting, ribonuclease protection, quantitative RT-PCR (e.g., using the TaqMan®, PE Applied Biosystems), or microarray analysis may be used to confirm that HisRS mRNA expression is reduced in cells treated with the nucleic acid modulator (e.g., Current Protocols in Molecular Biology (1994) Ausubel F M et al., eds., John Wiley & Sons, Inc., chapter 4; Freeman W M et al., Biotechniques (1999) 26:112-125; Kallioniemi O P, Ann Med 2001, 33:142-147; Blohm D H and Guiseppi-Elie, A Curr Opin Biotechnol 2001, 12:41-47). Protein expression may also be monitored. Proteins are most commonly detected with specific antibodies or antisera directed against either the HisRS protein or specific peptides. A variety of means including Western blotting, ELISA, or in situ detection, are available (Harlow E and Lane D, 1988 and 1999, supra).

### Secondary Assays

Secondary assays may be used to further assess the activity of a HisRS-modulating agent identified by any of the above methods to confirm that the modulating agent affects HisRS in a manner relevant to the SREBP pathway. As used herein, HisRS-modulating agents encompass candidate clinical compounds or other agents derived from previously identified modulating agent. Secondary assays can also be used to test the activity of a modulator on a particular genetic or biochemical pathway or to test the specificity of the modulator's interaction with HisRS.

Secondary assays generally compare like populations of cells or animals (e.g., two pools of cells or animals that endogenously or recombinantly express HisRS) in the presence and absence of the candidate modulator. In general, such assays test whether treatment of cells or animals with a candidate HisRS-modulating agent results in changes in the SREBP pathway, lipid metabolism, and/or adipogenesis, in comparison to untreated (or mock- or placebo-treated) cells or animals. Certain assays use sensitized genetic backgrounds, used herein to describe cells or animals engineered for altered expression of genes in the SREBP or interacting pathways, or other pathways associated with lipid metabolism and/or adipogenesis.

### Cell-Based Assays

Cell based assays may use a variety of mammalian cell types capable of SREBP signaling, including HEK-293 cells, CHO cells, primary hepatocytes, or hepatocytic cell lines such as McA-RH7777 (DeBose-Boyd et al., 2001, PNAS 98:1477-1482) or HEPG2 (Kotzka et al., 2000J. Lipid Res. 41:99-108). Cell based assays may detect endogenous SREBP pathway activity or may rely on recombinant expression of SREBP pathway components. Cell based assays typically use culture condition that permit SREBP signaling, such as low cholesterol or low PUFA conditions, or glucose- or insulin-stimulation. Candidate modulators are typically added to the cell media but may also be injected into cells or delivered by any other efficacious means.

In one embodiment, SREBP pathway activity is assessed by measuring expression of SREBP transcriptional targets. Many transcriptional targets are known (e.g., Osborne T F, 2001, J Biol Chem 275:32379-32382; Horton J D et al, 1998, J Clin Invest 101:2331-2339; Shimano H et al, 1997, J Clin Invest 100:2115-2124; Shimomura I et al, 1999, J Biol Chem 274: 30028-30032). Any available means for expression analysis, as previously described, may be used. Typically, mRNA expression is detected. In a preferred application, Taqman analysis is used to directly measure mRNA expression. Alternatively, expression-is indirectly monitored from a transgenic reporter construct comprising sequences encoding a reporter gene (such as luciferase, GFP or other fluorescent proteins, beta-galactosidase, etc.) under control of regulatory sequences (e.g., enhancer/promoter regions) of an SREBP transcriptional target gene. Methods for making and using reporter constructs are well known (e.g., Chakravarty K. et al., 2001J. Biol. Chem. 276:34816-34823).

In another embodiment, assays monitor SREBP processing events, such as cleavage of the membrane-bound form of SREBP, or nuclear translocation or nuclear accumulation of the activated form of SREBP. These events can be monitored directly by monitoring levels of membrane bound and cleaved forms of the protein. Typically, cells are fractionated, and protein levels in nuclear and membrane fractions are measured using immunohistochemistry. Alternatively, SREBP cleavage can be monitored indirectly using specific reporters for SREBP cleavage. In one example, a fusion construct comprising sequences encoding the signal peptide and soluble catalytic domain of alkaline phosphatase (AP) linked to the C-terminal (regulatory) domain of SREBP is introduced into cells. SREBP cleavage is monitored as secretion of AP, which is detected using a standard alkaline phosphatase assay (Sakai J., et al., 1998, Mol. Cell 2:505-514). In another example, a fusion construct is generated in which the transcriptional activator domain of SREBP is replaced with another transcriptional activator domain, such as yeast GAL4. The substituted domain, which is preferably from a different species, specifically activates transcription of a reporter gene under the control of responsive regulatory sequences, such as UAS if GAL4 is used.

In another embodiment, assays measure candidate modulators′ effects on the functional output of SREBP signaling, such as lipid accumulation and lipid metabolism. In one preferred application, lipid accumulation is measured by staining fixed cells with Oil Red O (Foretz et al., 1999, PNAS 96:12737-12742). In another preferred application, lipid synthesis is monitored by measuring C14 acetate incorporation into either cholesterol or fatty acids (Pai, J -T et al., 1998, J. Biol. Chem. 273:26138-26148).

### Animal Assays

A variety of non-human animal models of lipid metabolic disorders may be used to test candidate HisRS modulators. Such models typically use genetically modified animals that have been engineered to mis-express (e.g., over-express or lack expression in) genes involved in lipid metabolism, adipogenesis, and/or the SREBP pathway. Additionally, particular feeding conditions, and/or administration or certain biologically active compounds, may contribute to or create animal models of lipid and/or metabolic disorders. Assays generally required systemic delivery of the candidate modulators, such as by oral administration, injection (intravenous, subcutaneous, intraperitoneous), bolus administration, etc.

In one embodiment, assays use mouse models of diabetes and/or insulin resistance. Mice carrying knockouts of genes in the leptin pathway, such as ob (leptin) or db (leptin receptor), or the insulin signaling pathway, such as the insulin receptor (InR) or insulin receptor substrate (IRS), develop symptoms of diabetes, and show hepatic lipid accumulation (fatty liver) and, frequently, increased plasma lipid levels (Nishina et al., 1994, Metabolism 43:549-553; Michael et al., 2000, Mol Cell 6:87-97; Bruning J C et al., 1998, Mol Cell 2:559-569). Certain susceptible wild type mice, such as C57BIJ6, exhibit similar symptoms when fed a high fat diet (Linton and Fazio, 2001, Current Opinion in Lipidology 12:489-495). Accordingly, appropriate assays using these models test whether administration of a candidate modulator alters, preferably decreases lipid accumulation in the liver. Lipid levels in plasma and adipose tissue may also be tested. Methods for assaying lipid content, typically by FPLC or calorimetric assays (Shimano H et al., 1996, J Clin Invest 98:1575-1584: Hasty et al., 2001, J Biol Chem 276:37402-37408), and lipid synthesis, such as by scintillation measurement of incorporation of radio-labeled substrates (Horton J D et al., 1999, J Clin Invest 103:1067-1076), are well known in the art. Other useful assays test blood glucose levels, insulin levels, and insulin sensitivity (e.g., Michael M D, 2000, Molecular Cell 6: 87). Additionally, SREBP pathway activity may be tested by examining changes in the transcription of SREBP target genes in the liver. Exemplary target genes are associated with fatty acid metabolism and include acetyl CoA carboxylase, fatty acid synthase, ATP citrate lyase, glycerol-3-phosphate acyltransferase, glucose-6-phosphate dehydrogenase, malic enzyme, and stearoyl-CoA desaturase-1,etc. (Shimomura I et al, 1999, supra). Other target genes are associated with cholesterol metabolism and include HMG-CoA synthase, HMG-CoA reductase, squalene synthase, lipoprotein lipase, the low-density lipoprotein receptor (LDLR), etc. (Horton J D et al, 1998, supra).

Other appropriate animal models have specific alterations in SREBP pathway genes. For instance, mice that overexpress a constitutively active form of SREBP under control of the PEPCK promoter develop display fatty liver. In a low-density lipoprotein receptor (LDLR) null background, plasma lipids increase as well (Horton J D et al, 1999, J Clin Invest 103:10677-1076). Assays using these mice may measure both hepatic and plasma lipid levels.

In another embodiment, assays use mouse models of lipoprotein biology and cardiovascular disease. For instance, mouse knockouts of apolipoprotein E (apoE) display elevated plasma cholesterol and spontaneous arterial lesions (Zhang S H, 1992, Science 258:468-471). Transgenic mice over-expressing cholesterol ester transfer protein (CETP) also display increased plasma lipid levels (specifically, very-low-density lipoprotein [VLDL] and low-density lipoprotein [LDL] cholesterol levels) and plaque formation in arteries (Marotti K R et al., 1993, Nature 364:73-75). Assays using these models may test whether administration of candidate modulators alters plasma lipid levels, such as by decreasing levels of the pro-atherogenic LDL and VLDL, increasing HDL, or by decreasing overall lipid (including trigyceride) levels. Additionally histological analysis of arterial morphology and lesion formation (i.e., lesion number and size) may indicate whether a candidate modulator can reduce progression and/or severity of atherosclerosis. Numerous other mouse models for atherosclerosis are available, including knockouts of Apo-A1, PPARgamma, and scavenger receptor (SR)-B1 in LDLR- or ApoE-null background (reviewed in, e.g., Glass C K and Witztum J L, 2001, Cell 104:503-516).

In another embodiment, the ability of candidate modulators to alter plasma lipid levels and artherosclerotic progression are tested in mouse models for multiple lipid disorders. For instance, mice with knockouts in both leptin and LDL receptor genes display hypercholesterolemia, hypertriglyceridemia and arterial lesions and provide a model for the relationship between impaired fuel metabolism, increased plasma remnant lipoproteins, diabetes, and atherosclerosis (Hasty A H et al, 2001, supra.).

### Diagnostic Methods

The discovery that HisRS is implicated in the SREBP pathway provides for a variety of methods that can be employed for the diagnostic and prognostic evaluation of diseases and disorders associated with SREBP signaling and for the identification of subjects having a predisposition to such diseases and disorders. Any method for assessing HisRS expression in a sample, as previously described, may be used. Such methods may, for example, utilize reagents such as the HisRS oligonucleotides and antibodies directed against HisRS, as described above for: (1) the detection of the presence of HisRS gene mutations, or the detection of either over- or under-expression of HisRS mRNA relative to the non-disorder state; (2) the detection of either an over- or an under-abundance of HisRS gene product relative to the non-disorder state; and (3) the detection of perturbations or abnormalities in a biological pathway mediated by HisRS.

All references cited herein, including patents, patent applications, publications, and gene and sequence data accessible through the Genbank identifier numbers and websites provided, are incorporated in their entireties.

What is claimed is:

1. A method of identifying a candidate SREBP pathway modulating agent, said method comprising the steps of:
(a) providing an assay system comprising a HisRS polypeptide or nucleic acid;
(b) contacting the assay system with a test agent under conditions whereby, but for the presence of the test agent, the system provides a reference activity; and
(c) detecting a test agent-biased activity of the screening assay system, wherein a difference between the test agent-biased activity and the reference activity identifies the test agent as a candidate SREBP pathway modulating agent.

2. The method of claim 1 wherein the assay system includes a screening assay comprising a HisRS polypeptide and the candidate test agent is a small molecule modulator.

3. The method of claim 2 wherein the screening assay is an enzymatic assay.

4. The method of claim 1 wherein the assay system includes a binding assay comprising a HisRS polypeptide and the candidate test agent is an antibody.

5. The method of claim 1 wherein the assay system includes an expression assay comprising a HisRS nucleic acid and the candidate test agent is a nucleic acid modulator.

6. The method of claim 5 wherein the nucleic acid modulator is an antisense oligomer.

7. The method of claim 6 wherein the nucleic acid modulator is a PMO.

8. The method of claim 1 wherein the assay system comprises cultured cells or a non-human animal expressing HisRS, and wherein the assay system includes an assay that detects an agent-biased change in an activity associated with the SREBP pathway, lipid metabolism, or adipogenesis.

9. The method of claim 8 wherein the assay system comprises cultured cells.

10. The method of claim 9 wherein the assay detects an event selected from the group consisting of expression of SREBP transcriptional targets, SREBP protein processing, lipid accumulation, and lipid metabolism.

11. The method of claim 8 wherein the secondary assay system comprises a non-human animal.

12. The method of claim 11 wherein the non-human animal is a mouse providing a model of diabetes and/or insulin resistance.

13. The method of claim 11 wherein the non-human animal mis-expresses an SREBP pathway gene.

14. The method of claim 12 or 13 wherein the assay system includes an assay that detects an event selected from the group consisting of hepatic lipid accumulation, plasma lipid accumulation, adipose lipid accumulation, blood glucose level, insulin levels, insulin sensitivity, and expression of SREBP transcriptional targets.

15. The method of claim 11 wherein the non-human animal provides a model of atherosclerosis.

16. The method of claim 13 or 15 wherein the assay system includes an assay that detects an event selected from the group consisting of plasma lipid levels and arterial lesion formation.

17. The method of claim 16 wherein the assay system detects plasma lipid levels and the lipids detected are tricgycerides, cholesterol, or lipoproteins.

18. The method of claim 1, comprising the additional steps of:
(d) providing a second assay system comprising cultured cells or a non-human animal expressing HisRS,
(e) contacting the second assay system with the test agent of (b) or an agent derived therefrom under conditions whereby, but for the presence of the test agent or agent derived therefrom, the system provides a reference activity; and
(f) detecting an agent-biased activity of the second assay system, wherein a difference between the agent-biased activity and the reference activity of the second assay system confirms the test agent or agent derived therefrom as a candidate SREBP pathway modulating agent,
and wherein the second assay system includes a second assay that detects an agent-biased change in an acitivity associated with the SREBP pathway, lipid metabolism, or adipogenesis.

19. The method of claim 18 wherein the second assay system comprises cultured cells.

20. The method of claim 19 wherein the second assay detects an event selected from the group consisting of expression of SREBP transcriptional targets, SREBP protein processing, lipid accumulation, and lipid metabolism.

21. The method of claim 17 wherein the second assay system comprises a non-human animal.

22. The method of claim 21 wherein the non-human animal is a mouse providing a model of diabetes and/or insulin resistance.

23. The method of claim 21 wherein the non-human animal mis-expresses an SREBP pathway gene.

24. The method of claim 22 or 23 wherein the second assay system includes an assay that detects an event selected from the group consisting of hepatic lipid accumulation, plasma lipid accumulation, adipose lipid accumulation, blood glucose level, insulin levels, insulin sensitivity, and expression of SREBP transcriptional targets.

25. The method of claim 21 wherein the non-human animal provides a model of atherosclerosis.

26. The method of claim 23 or 25 wherein the assay system includes an assay that detects an event selected from the group consisting of plasma lipid levels and arterial lesion formation.

27. A method of modulating SREBP pathway activity in a mammalian cell comprising contacting the cell with an agent that specifically binds a HisRS polypeptide or nucleic acid.

28. The method of claim 27 wherein the agent is administered to a mammalian animal predetermined to have a pathology associated with the SREBP pathway.

29. The method of claim 27 wherein the agent is a small molecule modulator, a nucleic acid modulator, or an antibody.

30. The method of claim 27 wherein SREBP pathway activity is decreased.


SEQUENCE LISTING (2 sequences)

SEQ ID NO: 1 - DNA, 2334 bp, Homo sapiens
cacctggctttggcaacggaccgccccggtgcacgaagcgcagggcggtcgcaggagctggctgagcagcgaagcccaggccctcctgggaagaagtccgagcaggggcatcgcgcggcaggacgccggctttccgggacaggaacaaaaggcctgggaaggaggcgggtcagacaccaggaagggtggagggcactgcaaaggcggaacccgcgcaccagggaaaactcgggcgtttgcgcacctcactagtcacgaaggctgaggtgggatcctcccaagttcccttagtagccagcttcggcacttccgggaggagccggaaataatttttgtgctcggcggaggctctctaggcgtgcgacccagcgactcgatagccggaagtcatccttgctgaggctggggcaaccaccgcaggtcgagacagcaggcggctcaagtggacagccgggatggcagagcgtgcggcgctggaggagctggtgaaacttcagggagagcgcgtgcgaggcctcaagcagcagaaggccagcgccgagctgatcgaggaggaggtggcgaaactcctgaaactgaaggcacagctgggtcctgatgaaagcaaacagaaatttgtgctcaaaacccccaagggcacaagagactatagtccccggcagatggcagttcgcgagaaggtgtttgacgtaatcatccgttgcttcaagcgccacggtgcagaagtcattgatacacctgtatttgaactaaaggaaacactgatgggaaagtatggggaagactccaagcttatctatgacctgaaggaccagggcggggagctcctgtcccttcgctatgacctcactgttccttttgctcggtatttggcaatgaataaactgaccaacattaaacgctaccacatagcaaaggtatatcggcgggataacccagccatgacccgtggccgataccgggaattctaccagtgtgattttgacattgctgggaactttgatcccatgatccctgatgcagagtgcctgaagatcatgtgcgagatcctgagttcacttcagataggcgacttcctggtcaaggtaaacgatcgacgcattctagatgggatgtttgctatctgtggtgtttctgacagcaagttccgtaccatctgctcctcagtagacaagctggacaaggtgtcctgggaagaggtgaagaatgagatggtgggagagaagggccttgcacctgaggtggctgaccgcattggggactatgtccagcaacatggtggggtatccctggtggaacagctgctccaggatcctaaactatcccaaaacaagcaggccttggagggcctgggagacctgaagttgctctttgagtacctgaccctatttggcattgatgacaaaatctcctttgacctgagccttgctcgagggctggattactacactggggtgatctatgaggcagtgctgctacagaccccagcccaggcaggggaagagcccctgggtgtgggcagtgtggctgctggaggacgctatgatgggctagtgggcatgttcgaccccaaagggcgcaaggtgccatgtgtggggctcagcattggggtggagcggattttctccatcgtggaacagagactagaggctttggaggagaagatacggaccacggagacacaggtgcttgtggcatctgcacagaagaagctgctagaggaaagactaaagcttgtctcagaactgtgggatgctgggatcaaggctgagctgctgtacaagaagaacccaaagctactgaaccagttacagtactgtgaggaggcaggcatcccactggtggctatcatcggcgagcaggaactcaaggatggggtcatcaagctccgttcagtgacgagcagggaagaggtggatgtccgaagagaagaccttgtggaggaaatcaaaaggagaacaggccagcccctctgcatctgctgaactgaacaaactatcagaggaaaggaagtgggactggcactatttgaggttaagacaaactgcatatgtacttcaattgctttgcacttttccgtttcagcggaagacctgaagagtggtcagaacagagcctttgatttttattatggttattttattgattattactggcaaaaacggccaggtacaacacctttttcatacaaggcccaggaggcttagtccagtctgtgctcctgggctacaaggacccagcctgagatggtcccatctgcagggcccgcaccagttggagcagatacctccccaccaccaattgccaaaggtccaataaaatgcctcaaccacg

SEQ ID NO: 2 - protein, 509 aa, Homo sapiens
MAERAALEELVKLQGERVRGLKQQKASAELIEEEVAKLLKLKAQLGPDESKQKFVLKTPKGTRDYSPRQMAVREKVFDVIIRCFKRHGAEVIDTPVFELKETLMGKYGEDSKLIYDLKDQGGELLSLRYDLTVPFARYLAMNKLTNIKRYHIAKVYRRDNPAMTRGRYREFYQCDFDIAGNFDPMIPDAECLKIMCEILSSLQIGDFLVKVNDRRILDGMFAICGVSDSKFRTICSSVDKLDKVSWEEVKNEMVGEKGLAPEVADRIGDYVQQHGGVSLVEQLLQDPKLSQNKQALEGLGDLKLLFEYLTLFGIDDKISFDLSLARGLDYYTGVIYEAVLLQTPAQAGEEPLGVGSVAAGGRYDGLVGMFDPKGRKVPCVGLSIGVERIFSIVEQRLEALEEKIRTTETQVLVASAQKKLLEERLKLVSELWDAGIKAELLYKKNPKLLNQLQYCEEAGIPLVAIIGEQELKDGVIKLRSVTSREEVDVRREDLVEEIKRRTGQPLCIC